(19) United States
(12) Patent Application Publication
(10) Pub. No.: US 2024/0315651 A1
(43) Pub. Date: Sep. 26, 2024

(54) BIOMETRIC APPARATUS, BIOMETRIC METHOD, IMAGE PROCESSING METHOD, AND PROGRAM

(71) Applicants: Ryoji TAKEDA, Ishikawa (JP); Yoshihisa NAIJO, Ishikawa (JP); Akira KINOSHITA, Tokyo (JP); Yoshinori OKADA, Kanagawa (JP); Shigenori KAWABATA, Tokyo (JP)

(72) Inventors: Ryoji TAKEDA, Ishikawa (JP); Yoshihisa NAIJO, Ishikawa (JP); Akira KINOSHITA, Tokyo (JP); Yoshinori OKADA, Kanagawa (JP); Shigenori KAWABATA, Tokyo (JP)

(73) Assignees: Ricoh Company, Ltd., Tokyo (JP); National University Corporation Tokyo Medical and Dental University, Tokyo (JP)

(21) Appl. No.: 18/607,988

(22) Filed: Mar. 18, 2024

(30) Foreign Application Priority Data
Mar. 22, 2023 (JP) 2023-045661

(51) Int. Cl. A61B 6/00; A61B 5/05; A61B 6/46

(52) U.S. Cl. CPC A61B 6/4417; A61B 5/05; A61B 6/463; A61B 6/5247; A61B 2562/0223; A61B 2562/046

(57) ABSTRACT

A biometric apparatus including an irradiation device configured to deliver radiation to a subject, a radiation detector configured to perform radiation imaging on a test site of the subject, and a magnetic detector configured to detect biomagnetism of the subject, the magnetic detector being placed between the radiation detector and the irradiation device.

### CROSS-REFERENCE TO RELATED APPLICATIONS

The present application is based on and claims priority under 35 U.S.C. § 119 to Japanese Patent Application No. 2023-045661, filed Mar. 22, 2023, the contents of which are incorporated herein by reference in their entirety.

### BACKGROUND OF THE INVENTION

### 1. Field of the Invention

The present disclosure relates to a biometric apparatus, a biometric method, an image processing method, and a program.

### 2. Description of the Related Art

A biometric apparatus that measures weak biomagnetism generated from currents due to excitation of cells constituting the heart, spinal cord, peripheral nerve, and the like of a subject is known.

As the biometric apparatus, there is disclosed a biometric apparatus in which a radiation photoconductor is detachably attached thereto in order to superimpose a morphological image of a using subject the radiation photoconductor and information on biomagnetism based on a detection result of a biomagnetic detector to display the superimposed image (for example, see Patent Document 1).

In the apparatus of Patent Document 1, the subject is moved when a radiation detector such as the radiation photoconductor is attached or detached. Therefore, a mechanism for moving the subject is required. Furthermore, since the position of the subject may become misaligned during the movement, a case may arise where the morphological image and the information on the biomagnetism cannot be accurately superimposed on each other.

### RELATED-ART DOCUMENT

### Patent Document

[Patent Document 1] Japanese Patent No. 6513798

### SUMMARY OF THE INVENTION

According to an embodiment of the present disclosure, there is provided a biometric apparatus including an irradiation device configured to deliver radiation to a subject, a radiation detector configured to perform radiation imaging on a test site of the subject, and a magnetic detector configured to detect biomagnetism of the subject, the magnetic detector being placed between the radiation detector and the irradiation device.

### BRIEF DESCRIPTION OF THE DRAWINGS

FIG. 1 is a side view illustrating a configuration example of a biometric apparatus according to a first embodiment;

FIG. 2A is a cross-sectional view schematically illustrating a configuration example of a magnetic detector according to the first embodiment;

FIG. 2B is a cross-sectional view schematically illustrating a configuration example of a magnetic detector according to a modified example;

FIG. 3 is a diagram illustrating a hardware configuration example of a processing device according to the first embodiment;

FIG. 4 is a diagram illustrating a functional configuration example of the processing device according to the first embodiment;

FIG. 5 is a flowchart illustrating an example of processing of the biometric apparatus according to the first embodiment;

FIG. 6 is a diagram illustrating a state of radiation imaging in a state in which a subject is not placed;

FIG. 7 is a flowchart illustrating another example of the processing of the biometric apparatus according to the first embodiment;

FIG. 8 is a diagram illustrating an example of a second image according to the first embodiment;

FIG. 9 is a diagram illustrating an example of a first image according to the first embodiment;

FIG. 10 is a diagram illustrating an example of a third image according to the first embodiment;

FIG. 11 is a diagram illustrating an example of a fourth image according to the first embodiment;

FIG. 12 is a diagram illustrating a simulated subject in a radiation imaging experiment;

FIG. 13A is a diagram illustrating an example of a radiographic image in which a simulated subject and a magnetic detector are superimposed on each other;

FIG. 13B is a diagram illustrating an example of a second image including the magnetic detector but not including the simulated subject;

FIG. 13C is a diagram illustrating an example of a third image obtained by radiographing the simulation subject;

FIG. 14 is a side view illustrating a configuration example of a biometric apparatus according to a first modified example;

FIG. 15 is a side view illustrating a configuration example of a biometric apparatus according to a second modified example;

FIG. 16 is a side view illustrating a configuration example of a biometric apparatus according to a third modified example;

FIG. 17 is a side view illustrating a configuration example of a biometric apparatus according to a fourth modified example;

FIG. 18 is a side view illustrating a configuration example of a biometric apparatus according to a fifth modified example;

FIG. 19 is a diagram illustrating a functional configuration example of a processing device according to the second embodiment; and

FIG. 20 is a diagram for explaining brightness adjustment processing performed on each region obtained by divided by an adjusting part according to the second embodiment.

### DESCRIPTION OF THE EMBODIMENTS

The present disclosure has an object to provide a biometric apparatus in which a radiation detector can be attached and detached without moving a subject.

The biometric apparatus, a biometric method, and an image processing method according to each embodiment of the present disclosure will be described in detail with reference to the drawings. However, the following embodiments are merely examples of the biometric apparatus, the biometric method, and the image processing method for embodying the technical idea of the present embodiment, and the present disclosure is not limited the embodiments. Dimensions, materials, shapes, relative arrangements, and the like of the constituent parts described in the embodiments are not intended to limit the scope of the present disclosure thereto, and are merely examples for description, unless otherwise specified. In the following description, the same names and reference numerals denote the same or similar members, and detailed description thereof will be appropriately omitted.

In each drawing, orthogonal coordinates having an X axis, a Y axis, and a Z axis are used to represent directions. The Z-axis represents the vertical direction. The X-axis and the Y-axis represent two directions orthogonal to each other in a plane orthogonal to the Z-axis. A direction in which an arrow of the X axis is directed is represented as a +X direction, and a direction opposite to the +X direction is represented as a −X direction. A direction in which an arrow of the Y axis is directed is represented as a +Y direction, and a direction opposite to the +Y direction is represented as a −Y direction. A direction in which an arrow of the Z axis is directed is represented as a +Z direction, and a direction opposite to the +Z direction is represented as a −Z direction.

In the present specification, a top view refers to viewing an object from above (+Z direction). These directional expressions are for convenience of description and do not limit the directions of the embodiments of the present disclosure.

### First Embodiment

### <Overall Configuration Example of Biometric Apparatus 100>

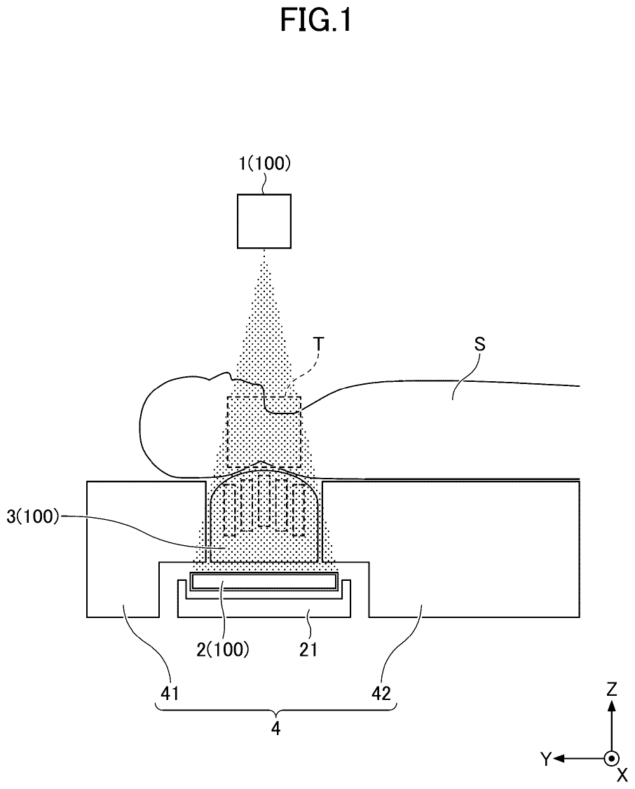

A configuration of a biometric apparatus 100 according to a first embodiment will be described with reference to FIG. 1. FIG. 1 is a diagram illustrating a configuration example of the biometric apparatus 100. FIG. 1 is a side view around the magnetic detector 3 as viewed from the +X direction.

As illustrated in FIG. 1, the biometric apparatus 100 includes an irradiation device 1, a radiation detector 2, and a magnetic detector 3. The biometric apparatus 100 detects weak biomagnetism generated from a subject S and outputs information on neuromuscular electrical action of f the spinal cord, peripheral nerve, heart, skeletal muscle, and the like of the subject S based on the biomagnetism.

The subject S corresponds to a living body. In the present specification, the subject S is a human; however, the subject S may be a living body other than a human. The subject S is placed on a table 4. The table 4 includes a head part table 41 and a body part table 42. The head part table 41 is used to support the head of the subject. The body part table 42 is a stand on which the body of the subject is placed.

In the present embodiment, the biometric apparatus 100 generates and outputs a superimposed image in which a morphological image of the subject S radiographed by the radiation detector 2 and information on biomagnetism based on a detection result of the magnetic detector 3 are superimposed. A diagnostician such as a doctor can easily identify which part of the body of the subject S the information on the biomagnetism corresponds to by observing the superimposed image. Note that the morphological image is an image including information on a morphological position of an organ of the subject S.

In order to generate the superimposed image, the biometric apparatus 100 performs radiation imaging and biomagnetism detection at different timings. The biometric apparatus 100 performs radiation imaging in a state where the radiation detector 2 is attached to the biometric apparatus 100. The biometric apparatus 100 performs biomagnetism detection in a state where the radiation detector 2 is detached from the biometric apparatus 100.

For example, when a radiation detector is attached to a biometric apparatus such that the radiation detector is placed between a subject and a magnetic detector, the subject may be required to be moved for securing a space or the like to attach the radiation detector. In order to move the subject S, a mechanism for moving the subject S is required. Furthermore, in order to move the subject S, the size of the biometric apparatus may need to be increased, or the apparatus configuration may become complicated. Also, when the subject S is moved, the position of the subject S may become misaligned during the movement, which may make it difficult to accurately superimpose the morphological image and the information on the biomagnetism.

In the present embodiment, the magnetic detector 3 is placed between the radiation detector 2 and the irradiation device 1. The subject S and the magnetic detector 3 are placed between the radiation detector 2 and the irradiation device 1. For example, the subject S is placed between the irradiation device 1 and the magnetic detector 3. By placing the magnetic detector 3 between the radiation detector 2 and the irradiation device 1, the radiation detector 2 can be placed on the opposite side of the subject S with the magnetic detector 3 interposed therebetween, and the radiation detector 2 need not be placed between the subject S and the magnetic detector 3. Thus, in the present embodiment, the radiation detector 2 can be attached and detached without moving the subject S. Furthermore, the need for a mechanism for moving the subject S can be eliminated, and the possibility of misalignment of the subject S can be reduced. The details of each component will be described below.

The irradiation device 1 delivers radiation to the subject S. The irradiation device 1 is placed above (in the +Z direction) the subject S placed on the table 4 and delivers radiation to the subject S. In the example illustrated in the present specification, the irradiation device 1 is an X-ray light source, and a plain X-ray can be delivered therefrom.

The radiation detector 2 is capable of radiographing a test site of the subject S. The radiation detector 2 has a light receiving surface for receiving radiation, and captures an image of the subject S with plain X-rays delivered from the irradiation device 1. The radiation detector 2 is provided independently of the table 4 at a position where an image of a test site T of the subject S can be captured. The radiation detector 2 acquires a captured image of the test site T by radiographing the test site T with the plain X-rays transmitted therethrough. The captured image is digital image data. The radiation detector 2 outputs information on the captured image to a processing device.

The radiation detector 2 is placed between the head part table 41 and the body part table 42. The radiation detector 2 is supported by a supporter 21 at a position where an image of the test site T of the subject S can be captured.

A flat panel detector or an imaging plate can be used as the radiation detector 2. The conversion method for the flat panel detector includes a direct conversion method, an indirect conversion method, and the like. The direct conversion method generates charges by a light receiving surface as a sensing element according to the dose of the delivered radiation, and converts the charges into electric signals. In the indirect method, the delivered radiation is converted into electromagnetic waves of another wavelength such as visible light, with a light receiving surface of a scintillator or the like. Then, according to the energy of the converted and delivered electromagnetic waves, charges are generated by a photoelectric conversion element such as a photodiode or the like to be converted into electric signals.

The imaging plate is a film coated with a photostimulable phosphor powder and housed in a casing called a cassette as a light receiving surface. The imaging plate is irradiated with the radiation that has transmitted through the test site T of the subject S, and the energy of the radiation is stored in the photostimulable phosphor. Subsequently, the imaging plate is irradiated with laser beams of a specific wavelength in a reading device, and by reading either the reflected light amount or the transmitted light amount of the delivered laser beams from the imaging plate is read, a photographed image can be acquired.

The magnetic detector 3 detects biomagnetism of the subject S. The magnetic detector 3 outputs information on the detected biomagnetism to the processing device. In the present embodiment, the magnetic detector 3 is placed between the radiation detector 2 and the irradiation device 1. The subject S is placed between the irradiation device 1 and the magnetic detector 3. The magnetic detector 3 is placed between the head part table 41 and the body part table 42 and is placed so as to face the test site T of the subject S.

### <Configuration Example of Magnetic Detector 3>

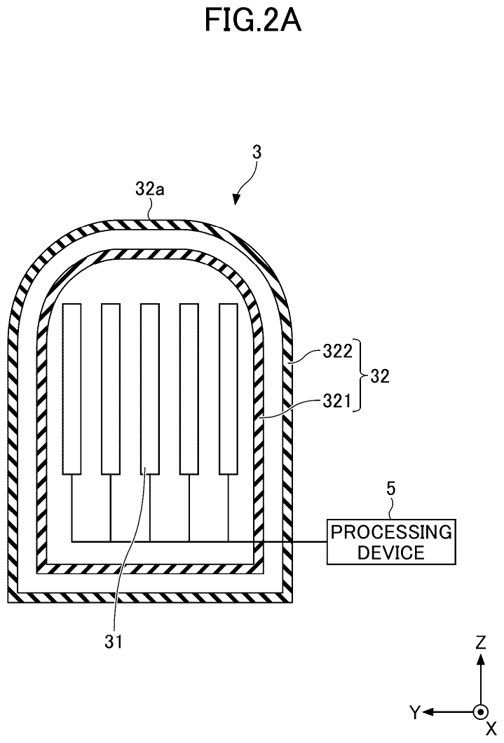

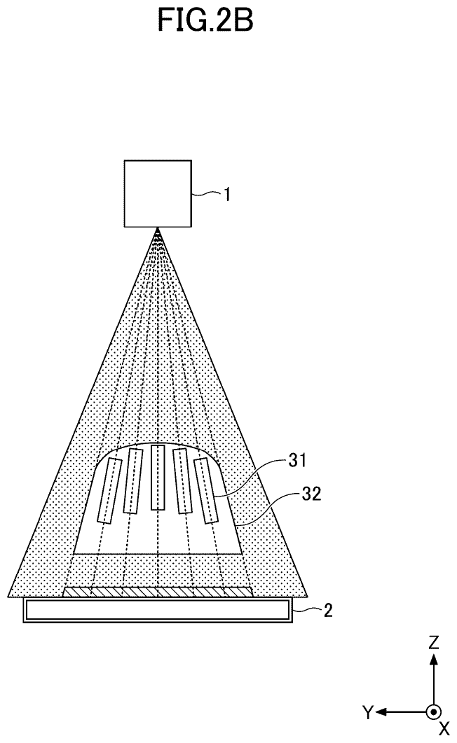

FIG. 2A is a cross-sectional view schematically illustrating a configuration example of the magnetic detector 3. FIG. 2A illustrates a cross section of the magnetic detector 3 cut along a virtual plane including the Y-axis and the Z-axis.

As illustrated in FIG. 2A, the magnetic detector 3 includes a magnetic sensor array in which magnetic sensors 31 for detecting biomagnetism are arranged in an array. The magnetic sensors 31 are held in a heat insulating container 32 having a temperature adjusting mechanism. The magnetic sensor 31 detects biomagnetism generated from the subject S. Specifically, examples of the magnetic sensor 31 include a superconducting quantum interference device (SQUID), an optically pumped atomic magnetometer (OPAM), a magnetoresistance effect device, a magneto-impedance device, and a fluxgate sensor. The SQUID sensor and the optically pumped atomic magnetometer have detection sensitivities capable of detecting extremely weak biomagnetism of approximately 10⁻¹⁸ T.

The heat insulating container 32 includes an inner tank 321 and an outer tank 322. The shape of the heat insulating container 32 is preferably such that a leading end surface 32a facing the subject S is shaped along the body surface of the test site T of the subject S, and the leading end surface may be flat or a curved. For example, as illustrated in FIG. 1, when biomagnetism measurement is performed by placing the cervical part of the subject S on the magnetic detector 3, the shape of the leading end surface 32a preferably has a curved surface shape that matches the circular arc of the cervical spinal cord.

A signal output from each magnetic sensor 31 is sent to a processing device 5 and is converted into magnetic information. By having the magnetic sensors 31, the magnetic detector 3 can acquire not only a large amount of magnetic information, but also detailed biometric information by two-dimensionally mapping the measured magnetic information or the like. Furthermore, when the magnetic sensor 31 can operate even at room temperature, the temperature adjusting mechanism and the heat insulating container 32 will be unnecessary. The number and the arrangement method of the magnetic sensors 31 are not particularly limited, and may be appropriately set according to the test site T of the subject S.

The magnetic sensor 31 includes a sensor bobbin around which a coil is wrapped. The sensor bobbin includes a hollow structure. The hollow structure is a structure having a cylindrical shape, a porous shape, or the like. The magnetic sensor 31 is preferably made of a low-density material to increase radiation transmissivity. The magnetic sensor 31 can increase the radiation transmissivity by including a low-density material, having a hollow structure, or reducing an occupancy area of the magnetic sensor on the leading end surface 32a of the heat insulating container 32 facing the subject S.

In a case where magnetic sensor 31 the operates at a low temperature, in order to prevent cracking due to cooling, the magnetic sensor 31 is preferably configured to include a material having a high heat conductivity and a low coefficient of thermal expansion for suppressing thermal shrinkage due to cooling. Examples of the material include polyether ether ketone (PEEK) resin, paper bakelite, cloth bakelite, polyphenylene sulfide (PPS) resin, polyoxymethylene (POM) resin, acrylic (poly methyl methacrylate (PMMA)) resin, and fiber reinforced plastics (FRP).

FIG. 2B is a cross-sectional view schematically illustrating a configuration example of the magnetic detector 3 according to a modified example. As illustrated in FIG. 2B, the magnetic sensors 31 the heat insulating container e arranged radially toward the center of the irradiation device 1, and thus it is possible to reduce the areas of the shadows appearing in an image Im1 captured by the radiation detector 2. It is desirable that both the sensor bobbins of the magnetic sensors 31 and the side surface parts of the heat insulating container 32 are also arranged radially toward the center of the irradiation device 1 as a focal point. The heat insulating container 32 is generally made of fiber reinforced plastic (FRP), metal stainless steel, or the like. Even if such a material having a low X-ray transmissivity is selected, the thickness of the container in the direction of the radiation is large and the container is captured darker in the captured image Im1, and thus the morphological information of the test site T superimposed thereon is likely to be lost. Therefore, it is desirable to minimize the shadow areas of the magnetic sensors 31 and the heat insulating container 32.

### <Configuration Example of Processing Device 5>

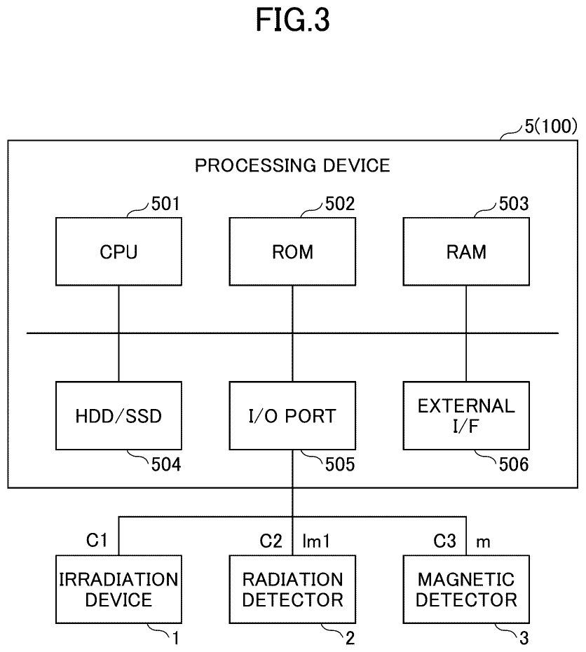

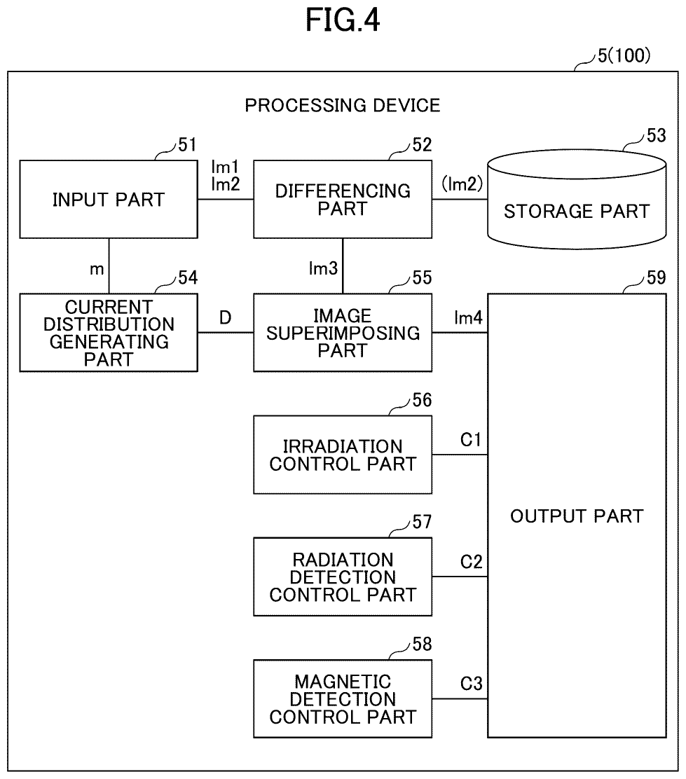

The biometric apparatus 100 may include a processing device in addition to the configuration of FIG. 1. In the present embodiment, the radiation detector 2 captures a first image including the subject S and the magnetic detector 3, and a second image including the magnetic detector 3 but not including the subject S. The processing device can generate a third image in which the magnetic detector 3 has been subjected to difference processing, based on the first image and the second image. Hereinafter, the configuration of the processing device according to the present embodiment will be described with reference to FIG. 3 and FIG. 4.

### (Hardware Configuration)

FIG. 3 is a block diagram illustrating an example of a hardware configuration of the processing device 5. The processing device 5 includes a central processing unit (CPU) 501, a read only memory (ROM) 502, a random access memory (RAM) 503, a hard disk drive/solid state drive (HDD/SSD) 504, a I/O port 505, and an external interface (I/F) 506. All of these components are interconnected via a system bus B.

The CPU 501 is a processor that executes control processing including various kinds of arithmetic processing. The ROM 502 is a non-volatile memory that stores a program used for driving the CPU 501, such as an initial program loader (IPL). The RAM 503 is a volatile memory used as a work area of the CPU 501. The HDD/SSD 504 is a non-volatile memory that stores various information such as programs, information detected by the radiation detector 2 and the magnetic detector 3, and the like.

The I/O port 505 is an input/output port that connects the irradiation device 1, the radiation detector 2, the magnetic detector 3, and the like to the processing device 5. The I/O port 505 can output an irradiation control signal C1 to the irradiation device 1, input magnetic information m that is from the magnetic detector 3, and input the image Im1 that is captured by the radiation detector 2.

The external I/F 506 is an interface for the processing device 5 to communicate with an external device of the biometric apparatus 100. The processing device 5 can communicate with an external computer or the like via the external I/F 506. The external I/F 506 can also receive operation input to the biometric apparatus 100 input by an operator of the biometric apparatus 100 using an operation part of the biometric apparatus 100.

### (Functional Configuration)

FIG. 4 is a block diagram illustrating an example of a functional configuration of the processing device 5. The processing device 5 includes an input part 51, a differencing part 52, a storage part 53, a current distribution generating part 54, an image superimposing part 55, an irradiation control part 56, a radiation detection control part 57, a magnetic detection control part 58, and an output part 59.

The functions of the input part 51 and the output part 59 are achieved by at least one of the I/O port 505 and the external I/F 506 illustrated in FIG. 3. The functions of the differencing part 52, the current distribution generating part 54, the image superimposing part 55, the irradiation control part 56, the radiation detection control part 57, and the magnetic detection control part 58 are achieved by the CPU 501 illustrated in FIG. 3 executing processing defined in the program stored in the ROM 502.

The input part 51 controls communication with the radiation detector 2 to input a radiograph from the radiation detector 2, the radiograph being captured by the radiation detector 2. The input part 51 controls communication with the magnetic detector 3 to input the magnetic information m from the magnetic detector 3, the information being detected by the magnetic detector 3.

In the present embodiment, the differencing part 52 acquires a third image Im3 in which the magnetic detector 3 is subjected to the difference processing, based on the first image Im1 including both the subject S and the magnetic detector 3 and a second image Im2 including the magnetic detector 3 but not including the subject S, and outputs the acquired third image. The first image Im1 and the second image Im2 are radiographs captured by the radiation detector 2.

For example, the differencing part 52 acquires, via the input part 51, the first image Im1 obtained by radiographing the radiation detector 2 and the irradiation device 1 with the subject S being placed therebetween. The differencing part 52 acquires, via the input part 51, the second image Im2 obtained by radiographing the radiation detector 2 and the irradiation device 1 without the subject S being placed therebetween. The differencing part 52 can generate the third image Im3 by performing image difference processing in which the second image Im2 is subtracted from the first image Im1.

In the present embodiment, the differencing part 52 may acquire, from the storage part 53, the second image Im2 that has been stored in the storage part 53. In such a case, the second image Im2 is radiographed in advance by the radiation detector 2 and stored in the storage part 53. It will be unnecessary for the biometric apparatus 100 to preform radiation imaging again in a state in which the subject S is not placed between the radiation detector 2 and the irradiation device 1. In FIG. 4, the second image Im2 is not included in data transmitted from the input part 51 to the differencing part 52. In the present embodiment, the second image Im2 is read and acquired from the storage part 53, and thus it is possible to omit the time and effort of imaging and to shorten the measurement time of the biometric apparatus 100.

The current distribution generating part 54 generates current distribution information D on a current distribution based on the magnetic information m from the magnetic detector 3. The current distribution information D is obtained by performing current source reconstruction based on the magnetic information m as the detection result of the magnetic detector 3. In the present embodiment, the information on the biomagnetism based on the detection result of the magnetic detector 3 includes the current distribution information D. Thus, biomagnetism can be visualized. The current distribution generating part 54 outputs the generated current distribution information D to the image superimposing part 55.

The image superimposing part 55 generates a fourth image Im4 corresponding to a superimposed image in which the current distribution information) from the current distribution generating part 54 and the third image Im3 are superimposed. The fourth image Im4 is information in which the detection result of the biomagnetism of the magnetic detector 3 and the morphological image of the test site T are associated with each other. The image superimposing part 55 outputs the generated fourth image Im4 via the output part 59.

The irradiation control part 56 outputs the first control signal C1 to the irradiation device 1 via the output part 59, thereby controlling the operation of the irradiation device 1.

The radiation detection control part 57 outputs a second control signal C2 to the radiation detector 2 via the output part 59, thereby controlling the operation of the radiation detector 2.

The magnetic detection control part 58 outputs a third control signal C3 to the magnetic detector 3 via the output part 59, thereby controlling the operation of the magnetic detector 3.

The output part 59 controls communication with an external device to output data or a signal from the processing device 5 to the external device. For example, the output part 59 outputs the fourth image Im4 to the display and causes the display to display the fourth image. However, the output part 59 is not limited to this, and may output the fourth image Im4 by another output method (for example, output to a memory, external transmission, or the like).

Each function of the processing device 5 can be achieved by one or multiple processing circuits. Here, the “processing circuit” in the present specification includes a processor programmed to execute each function by software, such as a processor implemented by an electronic circuit, and a device such as an application specific integrated circuit (ASIC), a digital signal processor (DSP), a field programmable gate array (FPGA), or a conventional circuit module designed to execute each function of the biometric apparatus 100 described above.

### <Operation Example of Biometric Apparatus 100>

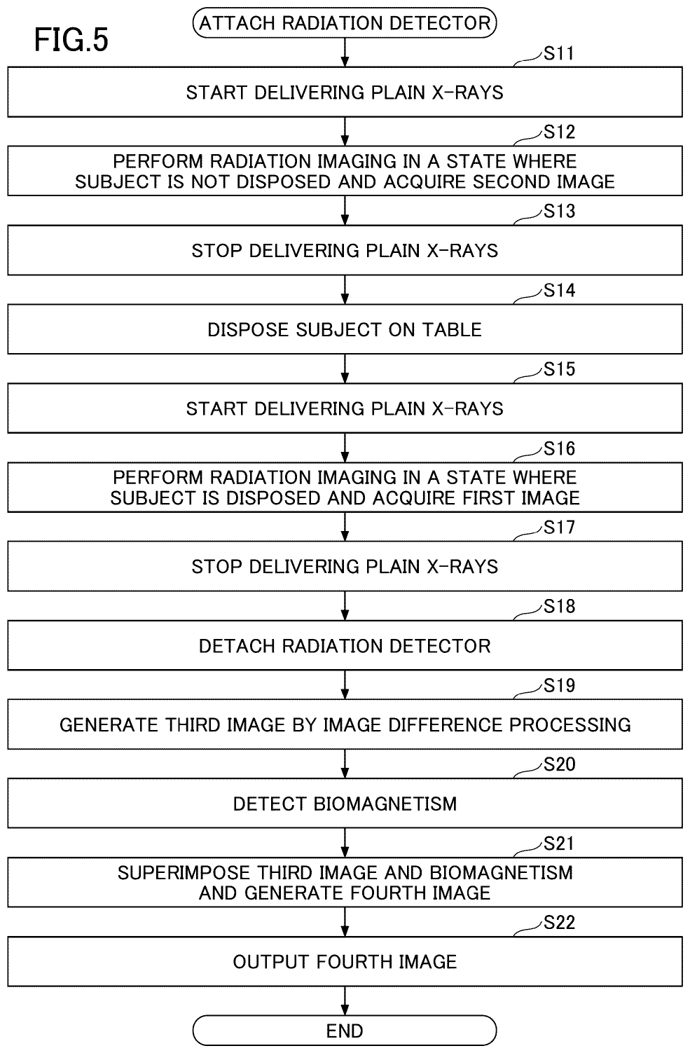

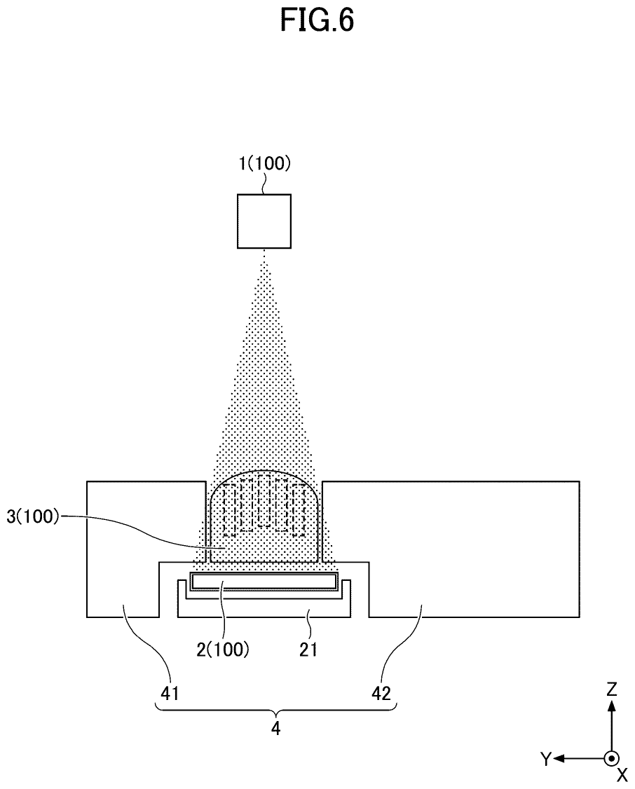

Operations of the biometric apparatus 100 will be described with reference to FIG. 5 to FIG. 7. Note that the description will also be made with reference to the functional configuration diagram of FIG. 4. FIG. 5 is a flowchart illustrating an example of the operations of the biometric apparatus 100. FIG. 6 is a diagram illustrating a state of radiation imaging in a state where the subject is not placed.

In FIG. 5, the biometric apparatus 100 starts the operations of FIG. 5 when the radiation detector 2 is attached to the biometric apparatus 100, for example.

In step S11, the biometric apparatus 100 controls the operation of the irradiation device 1 by the irradiation control part 56 to start irradiation of plain X-rays.

In step S12, the biometric apparatus 100 controls the operation of the radiation detector 2 by the radiation detection control part 57 to perform the radiation imaging in a state where the subject S is not placed and acquire the second image Im2. As illustrated in FIG. 6, the subject S is not placed between the irradiation device 1 and the radiation detector 2, and the magnetic detector 3 is placed therebetween.

In step S13, the biometric apparatus 100 controls the operation of the irradiation device 1 by the irradiation control part 56 to stop the delivering of the plain X-rays.

In step S14, the subject S is placed on the table 4 of the biometric apparatus 100.

In step S15, the biometric apparatus 100 controls the operation of the irradiation device 1 by the irradiation control part 56 to start the delivering of the plain X-rays.

In step S16, the biometric apparatus 100 controls the operation of the radiation detector 2 by the radiation detection control part 57 to perform the radiation imaging of the subject S being placed and acquire the first image Im1.

In step S17, the biometric apparatus 100 controls the operation of the irradiation device 1 by the irradiation control part 56 to stop the delivering of the plain X-rays.

In step S18, the radiation detector 2 is detached from the biometric apparatus 100.

In step S19, the biometric apparatus 100 performs the image difference processing on the first image Im1 and the second image Im2 by the differencing part 52 to generate the third image Im3.

In step S20, the biometric apparatus 100 controls the operation of the magnetic detector 3 by the magnetic detection control part 58 to detect the biomagnetism of the subject S. The biometric apparatus 100 also, generates, based on the magnetic information m from the magnetic detector 3, the current distribution information D on the current distribution by the current distribution generating part 54.

In step S21, the biometric apparatus 100 performs a process of superimposing the third image Im3 from the differencing part 52 and the current distribution information D from the current distribution generating part 54 by the image superimposing part 55, and generates the fourth image Im4.

In step S22, the biometric apparatus 100 outputs the fourth image Im4 to the external device by the output part 59.

As described above, the biometric apparatus 100 can generate the fourth image Im4 as the measurement result of the biomagnetism and output the generated fourth image.

In the present embodiment, when the radiation detector 2 is attached to the biometric apparatus 100 and when the radiation detector 2 is detached from the biometric apparatus 100, the subject S does not need to be moved. Therefore, in the present embodiment, it is possible to provide a biometric apparatus, a biological measurement method, and an image processing method in which the radiation detector 2 can be attached and detached without moving the subject.

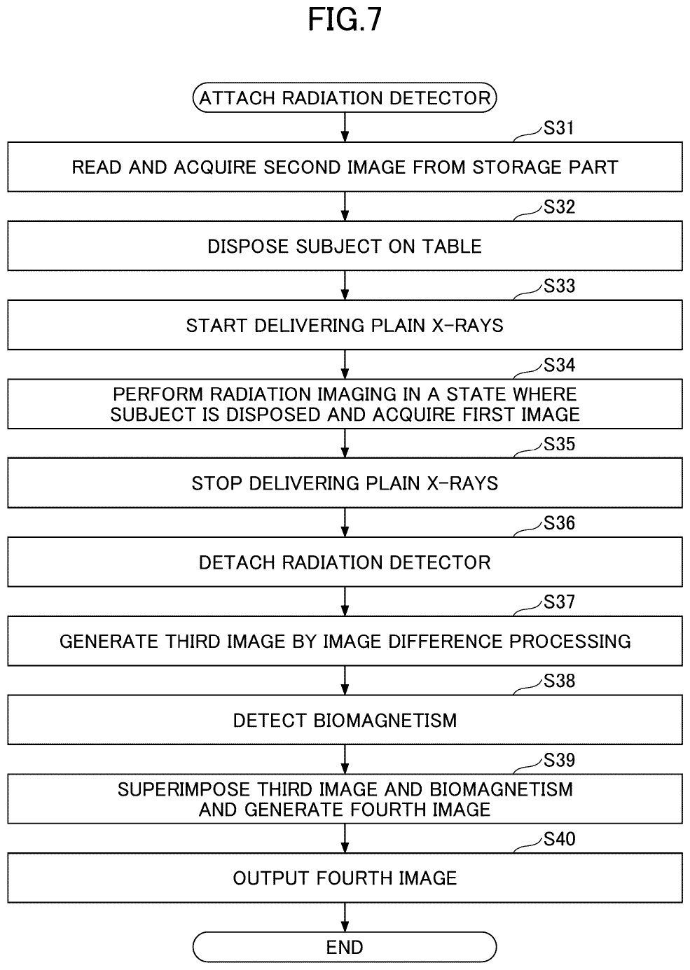

FIG. 7 is a flowchart illustrating another example of the operations of the biometric apparatus 100. The example illustrated in FIG. 7 is different from the example illustrated in FIG. 5 in that the biometric apparatus 100 reads and acquires the second image Im2 from the storage part 53.

In FIG. 7, the biometric apparatus 100 starts the operations of FIG. 7 at the timing when the radiation detector 2 is attached to the biometric apparatus 100, for example. Note that the second image Im2 is acquired in advance before the start of the operations and is stored in the storage part 53.

In step S31, the biometric apparatus 100 reads and acquires the second image Im2 stored in the storage part 53 by the differencing part 52. The operations after step S32 is the same as the operations after step S14 in FIG. 5, and thus the overlapping description will be omitted here. As described above, the biometric apparatus 100 can also generate and output the fourth image Im4 using the second image Im2 acquired from the storage part 53. Also, in such a case, the same result as that of the operations of FIG. 5 can be obtained.

### <Example of Acquisition Results for First to Fourth Images>

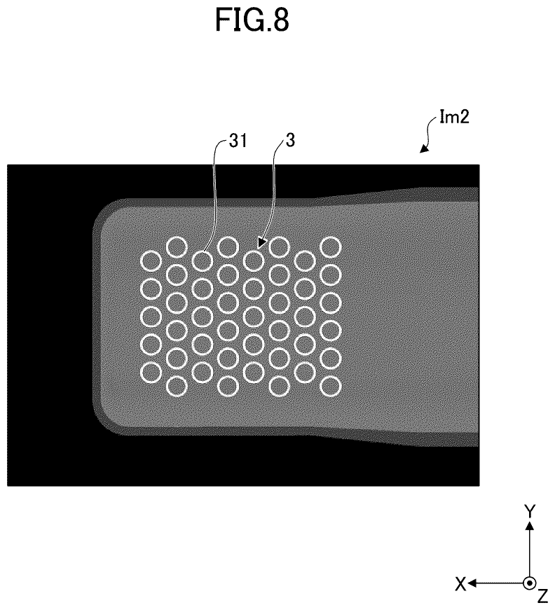

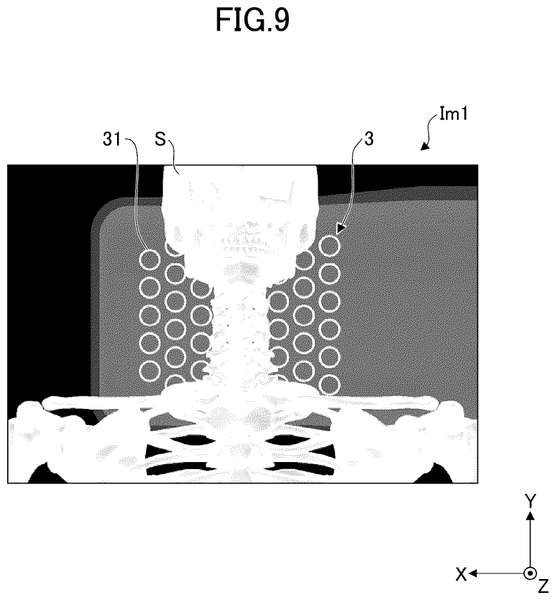

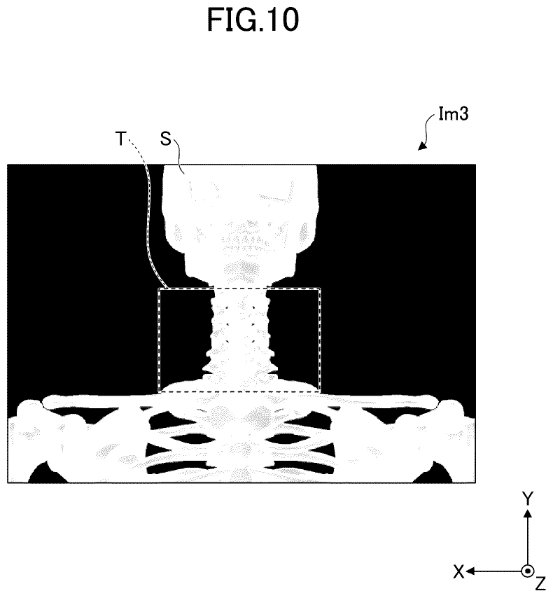

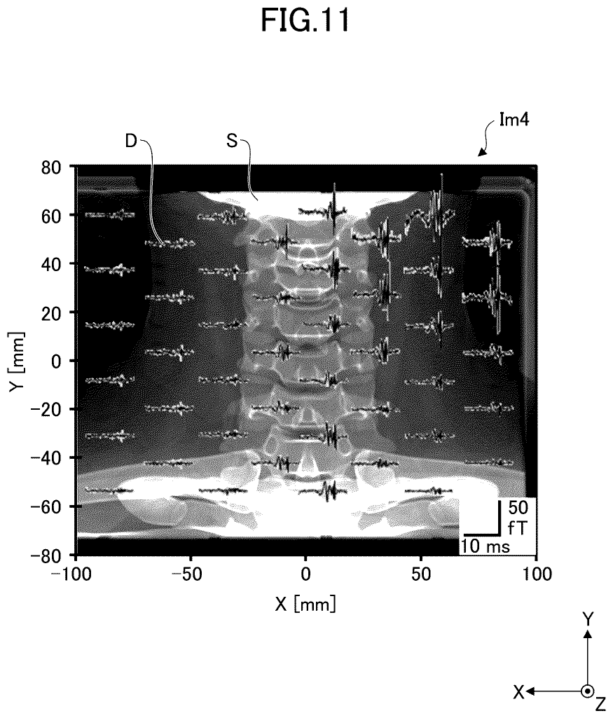

With reference to FIG. 8 to FIG. 11, the respective acquisition results of the first image Im1, the second image Im2, the third image Im3, and the fourth image Im4 will be described. FIG. 8 is a diagram illustrating an example of the second image Im2. FIG. 9 is a diagram illustrating an example of the first image Im1. FIG. 10 is a diagram illustrating an example of the third image Im3. FIG. 11 is a diagram illustrating an example of the fourth image Im4.

FIG. 8 illustrates the second image Im2 acquired in step S12 of FIG. 5 or step S31 of FIG. 7. As illustrated in FIG. 8, the second image Im2 includes the magnetic detector 3 and does not include the subject S. The second image Im2 is a plain X-ray image of the magnetic detector 3. The magnetic detector 3 includes the magnetic sensors 31 each having a substantially circular outline in a top view. In the claims and the present specification, an image including an image of a target such as the magnetic detector 3 may be expressed as an image including a target such as the magnetic detector 3.

FIG. 9 illustrates the first image Im1 acquired in step S16 of FIG. 5 or step S34 of FIG. 7. As illustrated in FIG. 9, the first image Im1 includes the magnetic detector 3 and the subject S. The first image Im1 is a plain X-ray image of the magnetic detector 3 and the subject S.

FIG. 10 illustrates the third image Im3 generated in step S19 of FIG. 5 or step S37 of FIG. 7. As illustrated in FIG. 9, the third image Im3 includes the subject S and does not include the magnetic detector 3. The third image Im3 is generated by performing the image difference processing in which the second image Im2 is subtracted from the first image Im1 by the differencing part 52 to remove the magnetic detector 3 from the first Note that the test site T in FIG. 10 indicates a target site for biomagnetic measurement.

FIG. 11 illustrates the fourth image Im4 generated in step S21 of FIG. 5 or step S39 of FIG. 7. The fourth image Im4 is an enlarged image of the region of the test site T in FIG. 10. As illustrated in FIG. 11, the fourth image Im4 includes the current distribution information D and the subject S. A diagnostician such as a doctor can easily identify which part of the body of the subject S the current distribution information D corresponds to by observing the fourth image Im4, thereby performing diagnosis.

In the example illustrated in the present specification, the third image Im3 corresponds to the morphological image of the subject S. By generating the fourth image Im4 using the third image Im3 as the morphological image, the diagnostician can perform diagnosis using the fourth image Im4 in which the magnetic detector 3 is not included, and thereby the diagnosis is facilitated. However, in the present embodiment, the first image Im1 may be used as a morphological image, and the fourth image Im4 may be generated by superimposing the first image Im1 and the current distribution information D. Also, in such a case, a result such that the radiation detector 2 can be attached and detached without moving the subject S can be obtained.

In the present embodiment, the measurement can be performed in a state where the subject S lies on the table 4. That is, the measurement can be performed even in a state where the subject S is less likely to move, as compared with a case where the measurement is performed in a state where the subject S is standing or sitting. This can prevent a decrease in measurement accuracy due to the movement of the subject S and improve the measurement accuracy.

In the present embodiment, the biometric apparatus 100 may include a guide member that is placed between the irradiation device 1 and the radiation detector 2 and can be subjected to radiation imaging. For example, a magnetic marker can be used as the guide member. When performing the image difference processing on the first image Im1 and the second image Im2, the biometric apparatus 100 can easily perform adjustment to match positions, magnifications, and the like of the first image Im1 and the second image Im2 by using each guide member included in the first image Im1 and the second image Im2 as guides. As a result of the adjustment of the positions, the magnifications, and the like of the first image Im1 and the second image Im2, the third image Im3 from which the magnetic detector 3 is appropriately removed can be generated.

### <Example of Radiation Imaging Experiment Results for Simulated Subject>

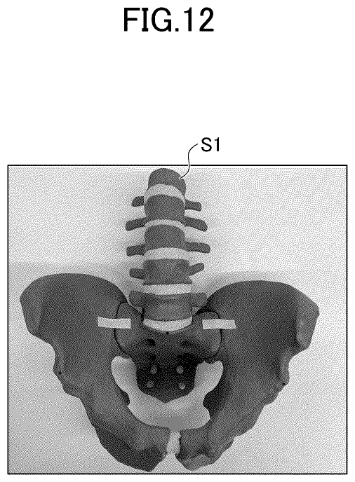

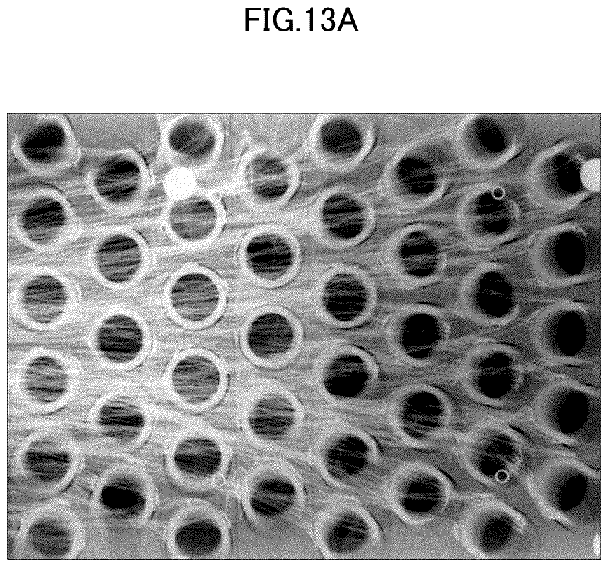

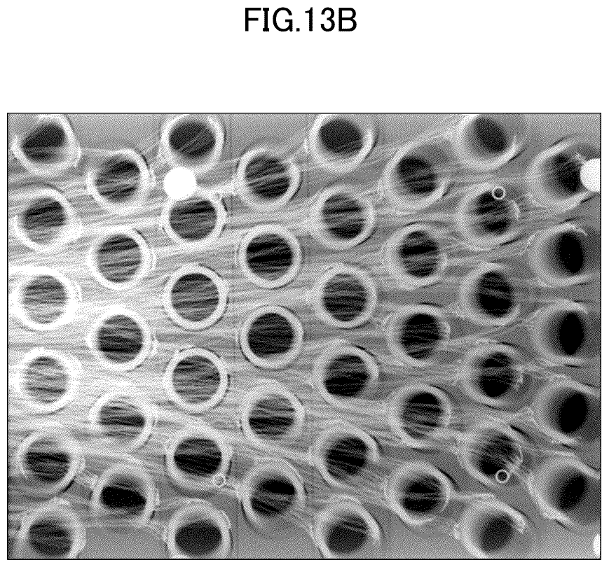

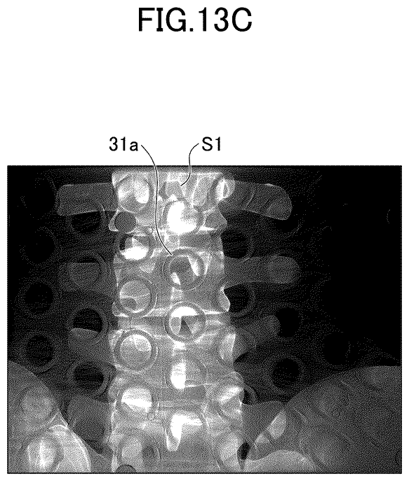

The radiation imaging experiment results for a simulated subject will be described with reference to FIG. 12 and FIG. 13A to FIG. 13C. FIG. 12 is a diagram illustrating an example of a simulated subject. FIG. 13A is a diagram illustrating an example of the first image Im1 obtained by radiographing the simulated subject.

As a simulated subject S1 illustrated in FIG. 12, a phantom simulating the periphery of the cervical vertebrae of a human for X-ray was used. In the arrangement illustrated in FIG. 1, 44 paper bakelite pipes each simulating a magnetic sensor were installed in the heat insulating container to simulate the magnetic detector 3, and radiation imaging was performed with plain X-rays. The pipes with an outer diameter of 20 mm, an inner diameter of 15 mm, and a length of 120 mm were used. The irradiation conditions of the plain X-rays were a tube voltage value of 80 kV and a mAs value of 24.6 mAs.

As illustrated in FIG. 13A, the radiographic image (first image Im1) in which the simulated subject S1 and the magnetic detector 3 are superimposed on each other is obtained. By performing the image difference processing by subtracting the second image Im2 including the magnetic detector 3 but not including the simulated subject S1 illustrated in FIG. 13B from the first image Im1 illustrated in FIG. 13A, the third image Im3, illustrated in FIG. 13C, including the simulated subject S1 and the magnetic detector 3 which had been subjected to the difference processing was generated.

### MODIFIED EXAMPLES

The arrangement of the irradiation device 1, the radiation detector 2, the magnetic detector 3, and the subject S in the biometric apparatus according to the embodiment can be modified in various ways. Hereinafter, modified examples will be described. Note that the same components as those in the above-described embodiment are denoted by the same reference numerals, and the overlapping description will be appropriately omitted. This point is the same in other modified examples and embodiments described below.

### First Modified Example

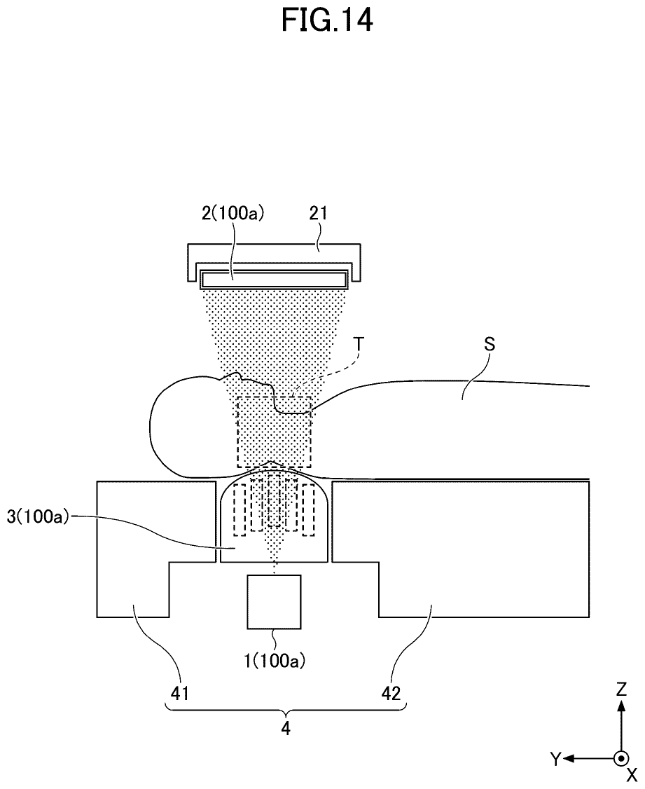

FIG. 14 is a side view illustrating configuration example of a biometric apparatus 100a according to a first modified example. The present modified example is different from the first embodiment mainly in that the subject S is placed between the magnetic detector 3 and the radiation detector 2. Furthermore, the present modified example is different from the first embodiment in that the radiation detector 2 is arranged above the subject S on the table 4 and the irradiation device 1 is arranged below the subject S.

In the present modified example, substantially the same result as that of the first embodiment can be obtained. Furthermore, since the magnetic detector 3 is placed between the irradiation device 1 and the subject S, the plain X-rays from the irradiation device 1 are attenuated by the magnetic detector 3, and thus it is possible to reduce the plain X-ray exposure dose to the subject S.

### Second Modified Example

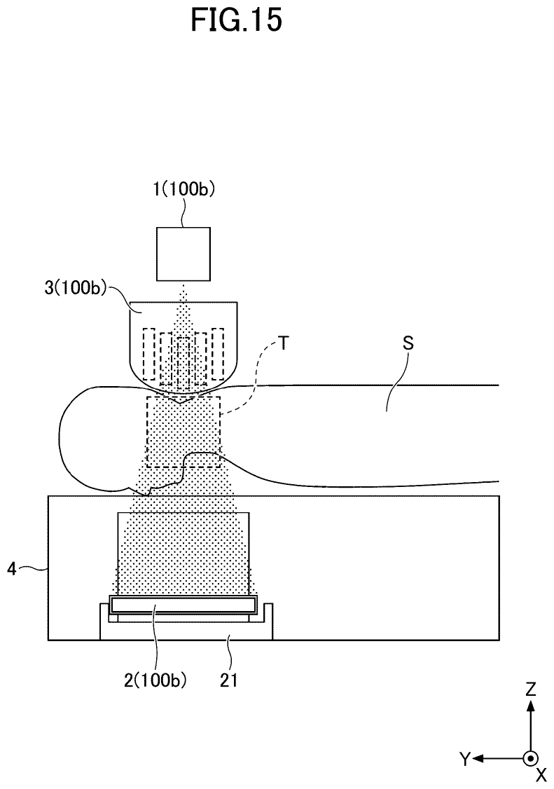

FIG. 15 is a side view illustrating an example of the configuration of a biometric apparatus 100b according to a second modified example. The present modified example is different from the first embodiment and the first modified example mainly in that the subject S is placed between the magnetic detector 3 and the radiation detector 2, the radiation detector 2 is placed below the subject S on the table 4, and the irradiation device 1 is placed above the subject S.

In the present modified example, substantially the same result as that of the first embodiment can be obtained. Furthermore, since the magnetic detector 3 is placed between the irradiation device 1 and the subject S, the plain X-rays from the irradiation device 1 are attenuated by the magnetic detector 3, and thus it is possible to reduce the plain X-ray exposure dose to the subject S.

In a case where the irradiation device 1 is placed below the subject, for example, the table 4 is raised in order to secure a distance for delivering radiation between the irradiation device 1 and the subject S, and consequently the subject S may experience strain from getting on and getting off the table 4. In the present modified example, since the irradiation device 1 is placed above the subject, the table 4 does not need to be raised in order to secure the distance for delivering radiation between the irradiation device 1 and the subject S. Therefore, it is possible to reduce the burden of moving up and down the table 4 on the subject S.

### Third Modified Example

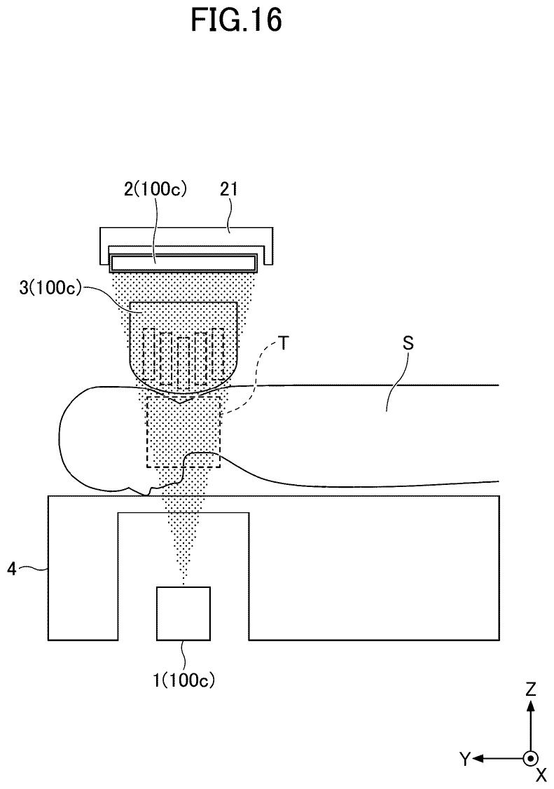

FIG. 16 is a side view illustrating a configuration example of a biometric apparatus 100c according to a third modified example. The present modified example is different from the first embodiment and the first and second modified examples mainly in that the subject S is placed detector between the magnetic 3 and the irradiation device 1, the irradiation device 1 is placed below the subject S on the table 4, and the detector 2 is placed above the subject S.

In the present modified example, substantially the same result as that of the first embodiment can be obtained.

### Fourth Modified Example

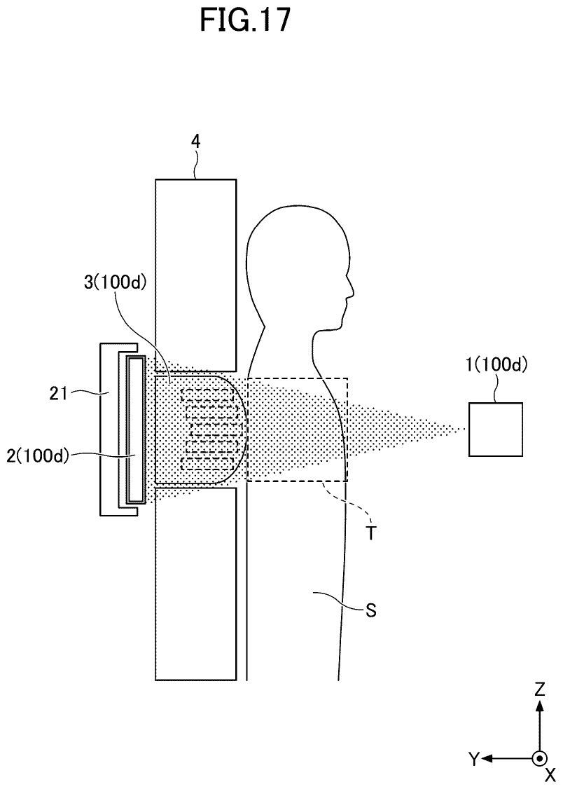

FIG. 17 is a side view illustrating a configuration example of a biometric apparatus 100d according to a fourth modified example. The present modified example is different from the first embodiment and the first to third modified examples mainly in that the subject S is placed between the magnetic detector 3 and the irradiation device 1, and measurement is performed in a state in which the subject S is standing or sitting.

Also, in the present modified example, substantially the same result as that of the first embodiment can be obtained except the result that is obtained when the measurement is performed in the state in which the subject S lies down. Furthermore, in the present modified example, since the table 4 does not need to be raised in order to secure the distance for delivering radiation between the irradiation device 1 and the subject S, it is possible to reduce the burden of moving up and down the table 4 on the subject S.

### Fifth Modified Example

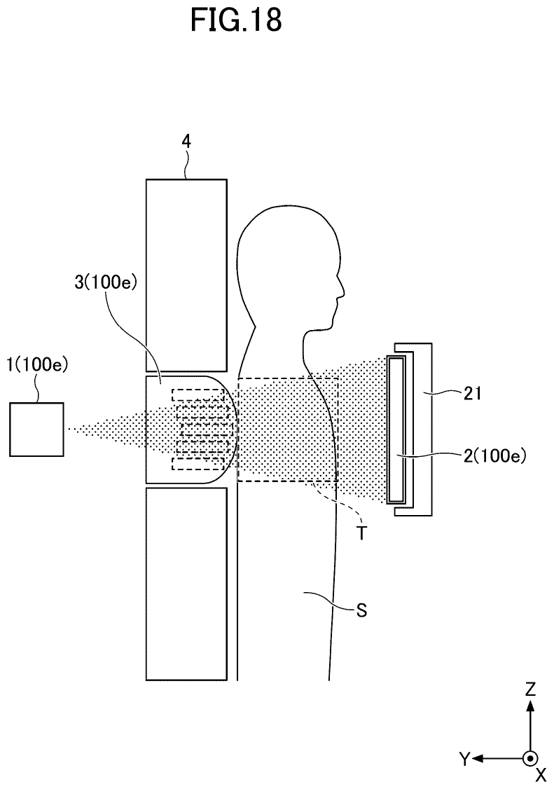

FIG. 18 is a side view illustrating a configuration example of a biometric apparatus 100e according to a fifth modified example. The present modified example is different from the first embodiment and the first to fourth modified examples mainly in that the subject S is placed between the magnetic detector 3 and the radiation detector 2, and measurement is performed in a state in which the subject S is standing or sitting.

In the present modified example, substantially the same result as that of the first embodiment can be obtained except the result that is obtained when the measurement is performed in the state in which the subject S lies down. Furthermore, in the present modified example, since the magnetic detector 3 is placed between the irradiation device 1 and the subject S, the plain X-rays from the irradiation device 1 are attenuated by the magnetic detector 3, and thus it is possible to reduce the plain X-ray exposure dose to the subject S. Also, in the present modified example, since the table 4 does not need to be raised in order to secure the distance for delivering radiation between the irradiation device 1 and the subject S, it is possible to reduce the burden of moving up and down the table 4 on the subject S.

### Second Embodiment

A biometric apparatus according to a second embodiment will be described. The present embodiment is mainly different from the first embodiment and the to first fifth modified examples in that the processing device includes an adjusting part that adjusts the brightness of the second image. The present embodiment is also different from the first embodiment and the first to fifth modified examples in that the processing device includes a complementing part. The complementing part complements an image region in which the morphological information of the subject is missing in the third image.

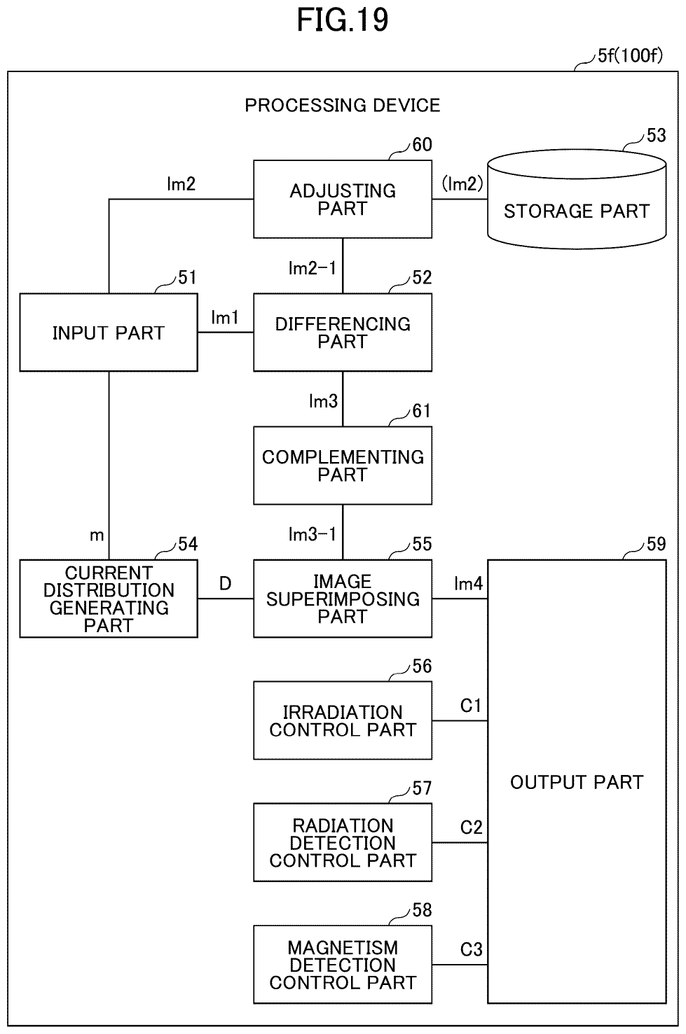

FIG. 19 is a block diagram illustrating a functional configuration example of a processing device 5f included in a biometric apparatus 100f according to the present embodiment. As illustrated in FIG. 19, the processing device 5f includes an adjusting part 60 and a complementing part 61.

The functions of the adjusting part 60 and the complementing part 61 are achieved by the CPU 501 illustrated in FIG. 3 executing the processing defined in the program stored in the ROM 502.

The adjusting part 60 adjusts the brightness of at least one of the first image Im1 or the second image Im2. In the example illustrated in FIG. 19, the brightness of the first image Im1 is adjusted. However, the adjusting part 60 may adjust the brightness of the second image Im2, or may adjust the brightness of each of the first image Im1 and the second image Im2. The adjusting part 60 outputs a second image Im2-1 obtained by performing brightness adjustment to the differencing part 52.

For example, the intensity of the radiation from the irradiation device 1 may differ between when the first image Im1 is acquired and when the second image Im2 is acquired, and a difference in the average brightness of the image may occur between the first image Im1 and the second image Im2. If there is such a difference in the average brightness, the image of the magnetic detector 3 cannot be appropriately removed when the image difference processing is performed on the first image Im1 and the second image Im2, and consequently the image of the magnetic detector 3 in the third image Im3 remains. From the viewpoint of facilitating the diagnosis by the diagnostician observing the fourth image Im4, it is preferable that the image of the magnetic detector 3 remaining in the third image Im3 is appropriately removed. In the present embodiment, the brightness of the second image Im2 is adjusted by the adjusting part 60, and thus the image of the magnetic detector 3 remaining in the third image Im3 can be minimized, and the image of the magnetic detector 3 can be appropriately removed.

The complementing part 61 complements the image region that is missing in the third image Im3 due to the removal of the magnetic detector 3 by the image difference processing performed on the first image Im1 and the second image Im2. For example, the complementing part 61 can complement the missing image region by performing interpolation processing such as linear interpolation or spline interpolation using pixels around the missing image region. The complementing part 61 outputs a third image Im3-1 obtained by performing complement processing to the image superimposing part 55. In the present embodiment, the missing image region is complemented by the complementing part 61, and thus the diagnostician can easily perform diagnosis by observing the fourth image Im4.

In the present embodiment, the adjusting part 60 may adjust the brightness of each image region obtained by dividing the second image Im2. For example, the brightness difference between the first image Im1 and the second image Im2 need not be uniform over the entire images, and may vary for each image region. In the present embodiment, the brightness is adjusted for each image region obtained by dividing the second image Im2, and thus the image of the magnetic detector 3 remaining in the third image Im3 can be appropriately removed.

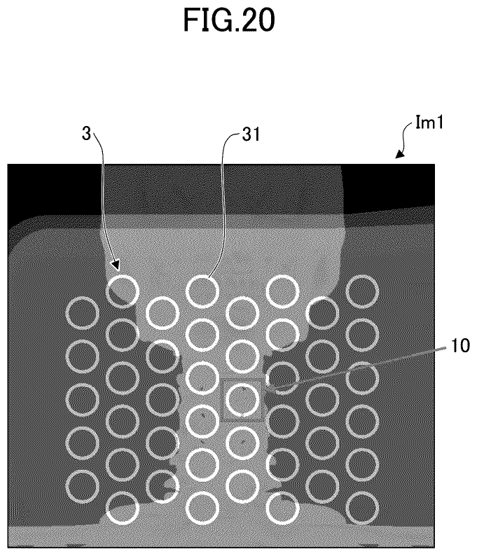

FIG. 20 is a diagram for explaining the brightness adjustment processing performed on each region obtained by divided by the adjusting part 60. In FIG. 20, a region 10 indicates one of the regions obtained by dividing the first image Im1. For example, the region division is performed so as to divide the first image Im1 including the magnetic sensors 31 into multiple regions each including one magnetic sensor.

The adjusting part 60 multiplies the average brightness of the pixels in the region 10 including one magnetic sensor 31 by a coefficient α. The adjusting part 60 generates the second image Im2-1 on which the brightness adjustment has been performed by combining the multiple regions 10. The coefficient α may be calculated by the following two methods, for example.
(1) Cut out the image into multiple regions 10, and generate a difference image for each region 10. Subject the generated difference images to Fourier transform, and obtain the sum of the power of the high-frequency region for the regions 10. Determine a coefficient α that minimizes the sum of the power of the high-frequency region by performing the above processing multiple times while changing the coefficient α.
(2) Cut out the image into multiple regions 10, and generate a difference image for each region 10. Obtain radial brightness distribution for each of the generated difference images to calculate the average by differentiating each brightness distribution. Set a position where the amount of change in the brightness distribution is maximized as a position of interest by executing the above processing while changing the coefficient α. Determine a coefficient α that minimizes the differential of the brightness distribution at the position of interest.

When the regions 10 are cut out, it is preferable that a position where the optical axes of the radiation detector 2 and the irradiation device 1 intersect is set as an origin, and the regions 10 each including one magnetic sensor 31 arranged close to the origin are cut out. The above process can also be performed by excluding some of the regions 10 in an area where the brightness is saturated or the like.

By dividing the image into image regions each corresponding to one magnetic sensor 31, the brightness adjustment for removing the magnetic sensor 31 can be optimized for each magnetic sensor 31, and thus each magnetic sensor 31 can be appropriately removed. However, the region division need not necessarily be performed to divide the image of the magnetic sensors 31 into multiple regions each including one magnetic sensor.

Although the preferred embodiments have been described in detail, the present disclosure is not limited to the above-described embodiments, and various modified examples and substitutions can be made to the above-described embodiments without departing from the scope of the appended claims.

The embodiments also include a biometric system including the above-described biometric apparatus 100. The biometric system may include an information processing apparatus such as a personal computer (PC), a display device, a storage device, and the like in addition to any one of the biometric apparatuses 100.

The present disclosure has, for example, the following aspects:

<1> A biometric apparatus including:

an irradiation device configured to deliver radiation to a subject;

a radiation detector configured to perform radiation imaging on a test site of the subject; and

a magnetic detector configured to detect biomagnetism of the subject, the magnetic detector being placed between the radiation detector and the irradiation device.

<2> The biometric apparatus according to above <1>, in which the irradiation device is placed apart from the magnetic detector such that the subject can be placed between the irradiation device and the magnetic detector.

<3> The biometric apparatus according to above <1>, in which the radiation detector is placed apart from the magnetic detector such that the subject can be placed between the radiation detector and the magnetic detector.

<4> The biometric apparatus according to any one of above <1> to <3>, in which the radiation detector is configured to acquire radiation image data in which the subject and the magnetic detector are superimposed.

<5> The biometric apparatus according to any one of above <1> to <4>, further including a guide member that is placed between the irradiation device and the radiation detector and can be subjected to the radiation imaging.

<6> The biometric apparatus according to any one of above <1> to <5>, in which the magnetic detector includes a plurality of magnetic sensors.

<7> The biometric apparatus according to any one of above <1> to <6>, in which

the magnetic detector includes a sensor bobbin around which a coil is wrapped, and

the sensor bobbin has a hollow structure.

<8> The biometric apparatus according to any one of above <1> to <7>, in which at least part of the magnetic detector is arranged radially toward a center of the irradiation device.

<9> The biometric apparatus according to any one of above <1> to <8>, in which

the radiation detector is configured to capture a first image including the subject and the magnetic detector and a second image including the magnetic detector but not including the subject,

the biometric apparatus further including:

circuitry; and

a memory storing computer-executable instructions that cause the circuitry to generate a third image in which the magnetic detector has been subjected to difference processing based on the first image and the second image.

<10> The biometric apparatus according to above <9>, in which the circuitry is caused to generate and output a fourth image in which information on the biomagnetism based on a detection result of the magnetic detector and the third image are superimposed on each other.

<11> The biometric apparatus according to above <10>, in which the information on the biomagnetism based on the detection result of the magnetic detector includes current distribution information obtained by performing current source reconstruction based on magnetic field information.

<12> The biometric apparatus according to any one of above <9> to <11>, in which the circuitry is caused to adjust brightness of at least one of the first image or the second image.

<13> The biometric apparatus according to above <12>, in which the circuitry is caused to adjust the brightness for each of a plurality of image regions obtained by dividing the second image.

<14> The biometric apparatus according to any one of above <9> to <13>, in which the circuitry is caused to complement an image region in which morphological information of the subject is missing in the third image.

<15> A biometric method executed by a biometric apparatus, in which the biometric apparatus includes:

an irradiation device configured to deliver radiation to a subject;

a radiation detector configured to output a second image captured in a state in which the subject is not placed between the radiation detector and the irradiation device, and a first image captured in a state in which the subject is placed between the radiation detector and the irradiation device;

a magnetic detector configured to detect biomagnetism of the subject, the magnetic detector being placed between the radiation detector and the irradiation device;

circuitry; and

a memory storing computer-executable instructions that cause the circuitry to:

generate a third image in which the magnetic detector has been subjected to difference processing based on the first image and the second image obtained by the radiation detector, the first image including the subject and the magnetic detector and the second image including the magnetic detector but not including the subject; and

generate and output a fourth image in which information on the biomagnetism based on a detection result of the magnetic detector and the third image are superimposed on each other.

<16> An image processing method of an image obtained by a biometric apparatus, in which the biometric apparatus includes:

an irradiation device configured to deliver radiation to a subject;

a radiation detector configured to perform radiation imaging on a test site of the subject;

a magnetic detector configured to detect biomagnetism of the subject, the magnetic detector being placed between the radiation detector and the irradiation device;

circuitry; and

a memory storing computer-executable instructions that cause the circuitry to:

generate a third image in which the magnetic detector has been subjected to difference processing based on a first image and a second image obtained by the radiation detector, the first image including the subject and the magnetic detector and the second image including the magnetic detector but not including the subject; and

generate and output a fourth image in which information on the biomagnetism based on detection result of the magnetic detector and the third image are superimposed on each other.

<17> A non-transitory computer-readable storage medium storing a program for causing an information processing apparatus to execute generating, based on a first image captured so as to include a subject and a magnetic detector and a second image captured so as to include the magnetic detector but not include the subject, a third image in which the magnetic detector has been subjected to difference processing.

According to the present disclosure, a biometric apparatus in which a radiation detector can be attached and detached without moving a subject can be provided.

What is claimed is:

1. A biometric apparatus comprising:
an irradiation device configured to deliver radiation to a subject;
a radiation detector configured to perform radiation imaging on a test site of the subject; and
a magnetic detector configured to detect biomagnetism of the subject, the magnetic detector being placed between the radiation detector and the irradiation device.

2. The biometric apparatus according to claim 1, wherein the irradiation device is placed apart from the magnetic detector such that the subject can be placed between the irradiation device and the magnetic detector.

3. The biometric apparatus according to claim 1, wherein the radiation detector is placed apart from the magnetic detector such that the subject can be placed between the radiation detector and the magnetic detector.

4. The biometric apparatus according to claim 1, wherein the radiation detector is configured to acquire radiation image data in which the subject and the magnetic detector are superimposed.

5. The biometric apparatus according to claim 1, further comprising a guide member that is placed between the irradiation device and the radiation detector and can be subjected to the radiation imaging.

6. The biometric apparatus according to claim 1, wherein the magnetic detector includes a plurality of magnetic sensors.

7. The biometric apparatus according to claim 1, wherein
the magnetic detector includes a sensor bobbin around which a coil is wrapped, and
the sensor bobbin has a hollow structure.

8. The biometric apparatus according to claim 1, wherein at least part of the magnetic detector is arranged radially toward a center of the irradiation device.

9. The biometric apparatus according to claim 1, wherein
the radiation detector is configured to capture a first image including the subject and the magnetic detector and a second image including the magnetic detector but not including the subject,
the biometric apparatus further comprising:
circuitry; and
a memory storing computer-executable instructions that cause the circuitry to generate a third image in which the magnetic detector has been subjected to difference processing based on the first image and the second image.

10. The biometric apparatus according to claim 9, wherein the circuitry is caused to generate and output a fourth image in which information on the biomagnetism based on a detection result of the magnetic detector and the third image are superimposed on each other.

11. The biometric apparatus according to claim 10, wherein the information on the biomagnetism based on the detection result of the magnetic detector includes current distribution information obtained by performing current source reconstruction based on magnetic field information.

12. The biometric apparatus according to claim 9, wherein the circuitry is caused to adjust brightness of at least one of the first image or the second image.

13. The biometric apparatus according to claim 12, wherein the circuitry is caused to adjust the brightness for each of a plurality of image regions obtained by dividing the second image.

14. The biometric apparatus according to claim 9, wherein the circuitry is caused to complement an image region in which morphological information of the subject is missing in the third image.

15. A biometric method executed by a biometric apparatus, wherein the biometric apparatus includes:
an irradiation device configured to deliver radiation to a subject;
a radiation detector configured to output a second image captured in a state in which the subject is not placed between the radiation detector and the irradiation device, and a first image captured in a state in which the subject is placed between the radiation detector and the irradiation device;
a magnetic detector configured to detect biomagnetism of the subject, the magnetic detector being placed between the radiation detector and the irradiation device;
circuitry; and
a memory storing computer-executable instructions that cause the circuitry to:
generate a third image in which the magnetic detector has subjected to been difference processing based on the first image and the second image obtained by the radiation detector, the first image including the subject and the magnetic detector and the second image including the magnetic detector but not including the subject; and
generate and output a fourth image in which information on the biomagnetism based on a detection result of the magnetic detector and the third image are superimposed on each other.

16. An image processing method of an image obtained by a biometric apparatus, wherein the biometric apparatus includes:
an irradiation device configured to deliver radiation to a subject;
a radiation detector configured to perform radiation imaging on a test site of the subject;
a magnetic detector configured to detect biomagnetism of the subject, the magnetic detector being placed between the radiation detector and the irradiation device;
circuitry; and
a memory storing computer-executable instructions that cause the circuitry to:
generate a third image in which the magnetic detector has been subjected to difference processing based on a first image and a second image obtained by the radiation detector, the first image including the subject and the magnetic detector and the second image including the magnetic detector but not including the subject; and
generate and output a fourth image in which information on the biomagnetism based on a detection result of the magnetic detector and the third image are superimposed on each other.

17. A non-transitory computer-readable storage medium storing a program for causing an information processing apparatus to execute generating, based on a first image captured so as to include a subject and a magnetic detector and a second image captured so as to include the magnetic detector but not include the subject, a third image in which the magnetic detector has been subjected to difference processing.
